SEQUENCE LISTING (2 sequences)

SEQ ID NO: 1 - DNA, 2143 bp, Homo sapiens
agtgcctttggttgctggagggaagaacacaatggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctgagtactctcatatgatgctgtgattttcctggagttgacagaaacacctatatttcccccagtcttccctgggagactactattaactgaaataaatactcagagcctgaaaaaaaaaaaaaaaaaa

SEQ ID NO: 2 - DNA, 1881 bp, Mus musculus
atgggtgtccagagaaggagcttccttcccgtgcttgtgctgagtgctctgctggctgtgggggccctagaaggatccaggaatcaggactggcttggtgtcccaagacaacttgtaactaaaacctggaacaggcagctgtaccccgagtggacagaggtgcaggggtctaactgctggagaggtggccaggtatctctgagggtcattaatgatgggcctacactggttggtgcaaatgcctccttttccattgccctgcacttccctggaagtcaaaaggtactaccggatggtcaggttatctgggccaacaacaccatcatcaatgggagccaggtgtggggaggacagccagtgtatccacaggagcctgatgatgcctgtgtcttccctgacggtggaccctgcccatctggtcctaaacctccgaagagaagctttgtttatgtttggaagacctggggaaaatactggcaagttctggggggtccagtgtccaggctgagcattgctacgggccacgcaaagctgggcacacacacaatggaagtgactgtctaccaccgacggggttcccagagctacgtgccccttgctcacgccagttcaaccttcaccattactgaccaggtacctttctccgtgagtgtgtcccagctacaggccttggacggagagaccaagcacttcctgagaaatcatcctctcatctttgcccttcagctccacgaccccagtggttatttggccgaggccgacctctcctacacatgggactttggagatggtactgggaccctgatctctcgggcacttgatgtcactcacacttacctggagtcgggctcagtcactgcccaggtggtactgcaggctgccattcctcttgtttcctgtggttcctccccagtcccgggtaccacagatggctacatgccaactgcagaagcacctggaaccacatctaggcaaggaaccactacaaaagttgtgggtactacacctggccagatgccaactacacagccctctggaaccacagttgtacaaatgccaaccacagaggtcacagctactacatctgagcagatgctgacctcagcggtcatagataccacactggcagaggtgtcaactacagagggtacaggtaccacacccacaaggccttctggaaccaccgttgcacaagcaacaaccacagagggtccagatgccagcccattgctgcccacacaaagttctacagggtccattagccctctactggatgacaccgacaccataatgcttgtgaagagacaagttcccctggactgtgttctatatcgatatggttctttctccctcgccctggacattgtccagggtattgaaagtgctgagatcctgcaggctgtgccattcagtgaaggggatgcatttgagctgactgtgtcctgccaaggcgggctacccaaggaagcctgtatggacatttcatcaccagggtgccagccccctgcccagaggctgtgccagtctgttccaccgagcccagactgccagctggttctacaccaagtgctgaaaggtggctcagggacatattgcctcaatgtgtctttggctgacgccaacagcctggcagtggccagcacccaacttgttgttcctggtcaagacggtggccttgggcaggctcccttgcttgtaggtatcttgctggtgttggtggctgtggtccttgcatctctgatacataggcatagacttaagaagcagggctcagtttcccaaatgccacatggtagcactcactggctgcgcctgcctccggtcttccgcgcccgcggccttggagaaaacagcccgctcctcagtggacagcaggtctga

(19) United States
(12) Patent Application Publication
(10) Pub. No.: US 2010/0068216 A1
(43) Pub. Date: Mar. 18, 2010

(54) Method and Compositions for Stimulation of an Immune Response to gp100 using a Xenogeneic gp100 Antigen

(75) Inventors: Alan N. Houghton, New York, NY (US); Clarissa C. Naftzger, Belmont, CA (US); Setaluri Vijayasaradhi, Madison, WI (US)

Correspondence Address: Larson & Anderson, LLC;re: MSK, P. O. BOX 4928, DILLON, CO, 80435-4928, US

(73) Assignee: SLOAN-KETTERING INSTITUTE FOR CANCER RESEARCH, New York, NY (US)

(21) Appl. No.: 12/622,087

(22) Filed: Nov. 19, 2009

Related U.S. Application Data
(63) Continuation-in-part of application No. 10/285,874, filed on Oct. 31, 2002.
Application No. 10/285,874 is a continuation-in-part of application No. 09/627,694, filed on Jul. 28, 2000, now abandoned.
Application No. 09/627,694 is a continuation-in-part of application No. 09/308,697, filed on May 21, 1999, now Pat. No. 6,328,969.
(60) Provisional application No. 60/036,419, filed on Feb. 18, 1997.

(51) Int. Cl. A61K 39/00; A61K 31/7088; A61P 35/00

(52) U.S. Cl. 424/184.1; 514/44 R

(57) ABSTRACT

Tolerance of the immune system for endogenous gp100 can be overcome and an immune response stimulated by administration of xenogeneic or xenoexpressed gp100 antigen. For example, mouse gp100, or antigenically-effective portions thereof, can be used to stimulate an immune response to the corresponding differentiation antigen in a human subject. Administration of xenogeneic antigens in accordance with the invention results in an effective immunity against gp100 expressed by the cancer in the treated individual, thus providing a therapeutic approach to the treatment of cancers expressing gp100, such as melanoma.

This application is a continuation-in-part of U.S. patent application Ser. No. 10/285,874, which is continuation-in-part of U.S. patent application Ser. No. 09/627,694, filed Jul. 28, 2000, which is continuation-in-part of U.S. patent application Ser. No. 09/308,697, filed May 21, 1999, which is a §371 National Phase of International Application No. PCT/US97/22669 filed Dec. 10, 1997. The application also claims benefit under 35 USC §119(e) of U.S. Provisional Application No. 60/036,419 filed Feb. 18, 1997. All of the aforementioned applications are incorporated herein reference.

### FIELD OF THE INVENTION

This application relates to a method and compositions for stimulation of an immune response to gp100.

### BACKGROUND OF THE INVENTION

Most tumor immunity is mediated by recognition of self-antigens, antigens present in cancer cells that are also found in normal host tissue. Houghton, A. N., J. Exp. Med. 180: 1-4 (1994). This type of immunity is more akin to autoimmunity than to immunity in infectious diseases, where the response is directed at a truly foreign antigen, present in the pathogen but not in host tissue. Evidence of this can be found in the autoimmune sequelae that often follow the development of successful tumor immunity. Bowne, W. B., et al., J. Exp. Med. 190(11):1717-1722 (1999).

Differentiation antigens form one prototype of self-antigens in cancer immunity. Houghton, A. N., et al., J. Exp. Med. 156(6):1755-1766 (1982). Differentiation antigens are tissue-specific antigens that are shared by autologous and some allogeneic tumors of similar derivation, and on normal tissue counterparts at the same stage of differentiation. Differentiation antigens have been shown to be expressed by a variety of tumor types, including melanoma, leukemia, lymphomas, colorectal, carcinoma, breast carcinoma, prostate carcinoma, ovarian carcinoma, pancreas carcinomas, and lung cancers. Typically the expression of these antigens changes as a cell matures and can characterize tumors as more or less differentiated. For example, differentiation antigens expressed by melanoma cells include Melan-A/MART-1, Pmel17, tyrosinase, gp75 and gp100. Differentiation antigens expressed by lymphomas and leukemia include CD19 and CD20/CD20 B lymphocyte differentiation markers. An example of a differentiation antigen expressed by colorectal carcinoma, breast carcinoma, pancreas carcinoma, prostate carcinoma, ovarian carcinoma, and lung carcinoma is the mucin polypeptide muc-1. A differentiation antigen expressed by breast carcinoma is her2/neu. The her2/neu differentiation antigen is also expressed by ovarian carcinoma. Differentiation antigens expressed by prostate carcinoma include prostate specific antigen, prostatic acid phosphatase, and prostate specific membrane antigen (PSMA).

Unfortunately, in most cases, the immune system of the individual is tolerant of these antigens, and fails to mount an effective immune response. For the treatment of cancers where the tumor expresses differentiation antigens therefore, it would be desirable to have a method for stimulating an immune response against the differentiation antigen in vivo. It is an object of the present invention to provide such a method.

### SUMMARY OF THE INVENTION

It has now been found that the tolerance of the immune system for endogenous gp100 can be overcome and an immune response stimulated by administration of xenogeneic gp100 and gp100 (including syngeneic gp100) expressed in cells of different species. For example, mouse gp100, or antigenically effective portions thereof, can be used to stimulate an immune response to the corresponding differentiation antigen in a human subject. Administration of xenogeneic or xenoexpressed antigens in accordance with the invention results in an effective immunity against gp100 expressed by the cancer in the treated individual, thus providing a therapeutic approach to the treatment of melanomas expressing gp100.

### BRIEF DESCRIPTION OF THE DRAWINGS

FIG. 1 shows lung metastases count in mice after immunization with hgp100 and mgp100.

FIGS. 2A-D show Kaplan-Meier survival curves, comparing immunization with hgp100 and mgp100, as well as various doses and combinations with GM-CSF DNA adjuvant.

FIG. 3 shows depigmentation by quadrant count of mice after DNA vaccination.

FIGS. 4A-B show IFN-γ release by CD8+ cells from immunized mice in response to gp100 peptide fragment, obtained by Elispot assay.

FIG. 5 shows representative flow cytometry dot plots, showing the change in gp100 fragment-reactive CD8+ cells at Baseline, Cross-over, and Post-Vaccination in human melanoma patients.

FIG. 6 shows representative flow cytometry dot plots illustrating the chemokine receptor 7, CD45RA, CD27, and CD28 subtypes of CD8+ cells in human melanoma patients.

FIG. 7 shows flow cytometry dot plots showing intracellular cytokine staining in the human melanoma patient that showed an increase in CD8+ IFN-γ+ cells after vaccination.

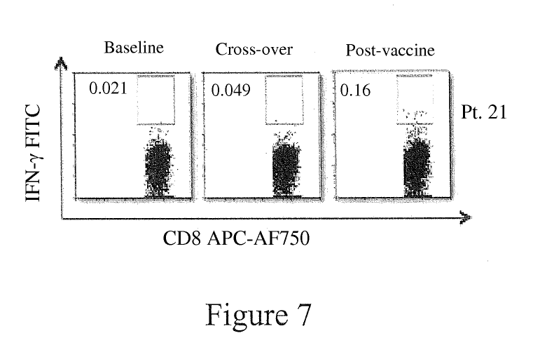

### DETAILED DESCRIPTION OF THE INVENTION

The present invention provides a method for stimulating an immune response to a tissue expressing gp100 in a subject individual. The subject individual is preferably human, although the invention can be applied in veterinary applications to animal species, preferably mammalian (for example horse, dog or cat) or avian species, as well.

As used in the specification and claims of this application, the term “immune response” encompasses both cellular and humoral immune responses. Preferably, the immune response is sufficient to provide immunoprotection against growth of tumors expressing gp100. The term “stimulate” refers to the initial stimulation of a new immune response or to the enhancement of a pre-existing immune response.

In accordance with the invention, a subject individual is treated to stimulate an immune response to endogenous gp100 by administering a xenogeneic or xenoexpressed gp100 antigen. The term “xenogeneic” denotes the fact that the administered antigen has a sequence peptide different from the gp100 of the species being treated and originates from a different species. For treatments of humans, preferred xenogeneic antigens will be rodent antigens, for example mouse, but could come from other mammals such as dog, cat, cow, or sheep, or from birds, fish, amphibian, reptile, insect or other more distantly related species. The term “xenoexpressed” refers to an antigen which may be syngeneic with the subject individual, but which is expressed in cells of a species different from the subject individual, for example in insect cells.

The term “gp100 antigen” refers to a protein/peptide antigen or to a polynucleotide having a sequence that is expressed in vivo to produce the protein/peptide antigen. In either case, the protein/peptide antigen may be the entire gp100 molecule, or some antigenic portion thereof derived from the extracellular domain. For example, as described below, plasmids were prepared using either full length cDNA or using a truncated portion encoding an amino acid strand (for example, amino acids 209-217 in the mouse protein).

Administration of a protein/peptide xenogeneic or xenoexpressed gp100 antigen can be accomplished by several routes. First, the xenogeneic gp100 may be administered as part of a vaccine composition which may include one or more adjuvants such as alum, QS21, TITERMAX or its derivatives, incomplete or complete Freund's and related adjuvants, and cytokines such as granulocyte-macrophage colony stimulating factor (GM-CSF), flt-3 ligand, interleukin-2, interleukin-4 and interleukin-12 for increasing the intensity of the immune response. The vaccine composition may be in the form of xenogeneic gp100 antigen in a solution or a suspension, or the gp100 antigen may be introduced in a lipid carrier such as a liposome. Such compositions will generally be administered by subcutaneous, intradermal or intramuscular route.

Vaccine compositions containing protein/peptide xenogeneic or xenoexpressed gp100 antigen are administered in amounts which are effective to stimulate an immune response to the target differentiation antigen in the subject individual. The preferred amount to be administered will depend on the species of the target individual and on the specific antigen, but can be determined through routine preliminary tests in which increasing doses are given and the extent of antibody formation or T cell response is measured by enzyme-linked immunosorbent assay (ELISA) or similar tests. T cell responses may also be measured by cellular immune assays, such as cytokine release assays and proliferation assays.

Xenogeneic gp100 antigen may also be introduced in accordance with the invention using a DNA immunization technique in which DNA encoding the antigen is introduced into the subject such that the antigen is expressed by the subject. Xenogeneic gp100 antigen may also be administered as a purified protein. Proteins can be purified for this purpose from cell lysates using column chromatography procedures. Proteins for this purpose may also be purified from recombinant sources, such as bacterial or yeast clones or mammalian or insect cell lines expressing the desired product.

Xenogeneic gp100 antigen may also be administered indirectly through genetic immunization of the subject with DNA encoding the antigen. cDNA encoding the xenogeneic gp100 antigen is combined with a promoter which is effective for expression of the cDNA in mammalian cells. This can be accomplished by digesting the nucleic acid polymer with a restriction endonuclease and cloning into a plasmid containing a promoter such as the SV40 promoter, the cytomegalovirus (CMV) promoter or the Rous sarcoma virus (RSV) promoter. The resulting construct is then used as a vaccine for genetic immunization. The cDNA can also be cloned into plasmid and viral vectors that are known to transduce mammalian cells. These vectors include retroviral vectors, adenovirus vectors, vaccinia virus vectors, pox virus vectors and adenovirus-associated vectors.

Xenogeneic antigen may also be administered in combination with anti-GITR (glucocorticoid-induced tumor necrosis factor receptor family gene), as described in Cohen A, et al, Agonist Anti-GITR Antibody Enhances Vaccine-Induced CD8+ T-cell Responses and Tumor Immunity, 66 CANCER RES. 4904 (2006).

The nucleic acid constructs containing the promoter, gp100 antigen-coding region and intracellular sorting region can be administered directly or they can be packaged in liposomes or coated onto colloidal gold particles prior to administration. Techniques for packaging DNA vaccines into liposomes are known in the art, for example from Murray, ed., GENE TRANSFER AND EXPRESSION PROTOCOLS, Humana Pres, Clifton, N.J. (1991). Similarly, techniques for coating naked DNA onto gold particles are taught in Yang, Gene transfer into mammalian somatic cells in vivo, CRIT. REV. BIOTECH. 12: 335-356 (1992), and techniques for expression of proteins using viral vectors are found in Adolph, K. ed., VIRAL GENOME METHODS, CRC Press, Florida (1996).

For genetic immunization, the vaccine compositions are preferably administered intradermally, subcutaneously or intramuscularly by injection or by gas driven particle bombardment, and are delivered in an amount effective to stimulate an immune response in the host organism. The compositions may also be administered ex vivo to blood or bone marrow-derived cells (which include APCs) using liposomal transfection, particle bombardment or viral infection (including co-cultivation techniques). The treated cells are then reintroduced back into the subject to be immunized. While it will be understood that the amount of material needed will depend on the immunogenicity of each individual construct and cannot be predicted a priori, the process of determining the appropriate dosage for any given construct is straightforward. Specifically, a series of dosages of increasing size, starting at about 0.1 μg is administered and the resulting immune response is observed, for example by measuring antibody titer using an ELISA assay, detecting CTL response using a chromium release assay or detecting TH (helper T cell) response using a cytokine release assay.

In accordance with a further aspect of the present invention, an immune response against a gp100 antigen can be stimulated by the administration of syngeneic gp100 antigen expressed in cells of a different species, i.e. by xenoexpressed gp100 antigen. In general, the subject being treated will be a human or other mammal. Thus, insect cells are a preferred type of cells for expression of the syngeneic differentiation antigen. Suitable insect cell lines include Sf9 cells and Schneider 2 Drosophila cells. The therapeutic differentiation antigen could also be expressed in bacteria, yeast or mammalian cell lines such as COS or Chinese hamster ovary cells. Host cells which are evolutionarily remote from the subject being treated, e.g. insects, yeast or bacteria for a mammalian subject, may be preferred since they are less likely to process the expressed protein in a manner identical to the subject.

To provide for expression of the differentiation antigen in the chosen system, DNA encoding the differentiation antigen or a portion thereof sufficient to provide an immunologically effective expression product is inserted into a suitable expression vector. There are many vector systems known which provide for expression of incorporated genetic material in a host cell, including baculovirus vectors for use with insect cells, bacterial and yeast expression vectors, and plasmid vectors (such as psvk3) for use with mammalian cells. The use of these systems is well known in the art.

For treatment of humans with a syngeneic differentiation antigen, cDNA encoding the human differentiation antigen to be targeted must be available. cDNA is produced by reverse transcription of mRNA, and the specific cDNA encoding the gp100 antigen can be identified from a human cDNA library using probes derived from the protein sequence of the differentiation antigen, which is known in the art, examples of which are found in Seq. ID No. 1 which follows the Examples.

Xenoexpressed gp100 antigen, like purified xenogeneic gp100 antigen, is administered to the subject individual in an amount effective to induce an immune response. The composition administered may be a lysate of cells expressing the xenoexpressed antigen, or it may be a purified or partially purified preparation of the xenoexpressed antigen.

The invention will now be further described with reference to the following, non-limiting examples:

### Example 1

Plasmid constructs were created by cloning human gp100 (hgp100) cDNA (2.1 kb) and mouse gp100 (mgp100) cDNA (1.9 kb) into pWRG1644 and pWRG7077 respectively. Plasmid constructs were coated onto 1-μm gold microcarriers for use in gene gun immunization. The gold-DNA complex was delivered to immunized C57BL/6 mice by helium driven gene gun for a total of 4 injections in each abdominal quadrant. The injections were repeated weekly from 0 to 5 times.

Tumor protection was assessed in two systems—intravenous and intradermal challenge.

To study lung metastasis, mice were injected intravenously by tail vein with B16 melanoma cells. Mice were killed after 14 days, and the lungs dissected and surface lung metastases were counted.

For the intradermal tumor experiments, mice were injected intradermally with B16 melanoma cells on the right flank 5 days after the final immunization. The mice were palpated for the presence of tumors, and average tumor diameter was measured with calipers every other day. Tumors were scored as present once they reached an average diameter of 2 mm. Mice were killed if tumors reached an average diameter of 2 cm or if they ulcerated. Kaplan-Meier tumor-free survival curves were constructed and log rank statistical analysis was performed to determine significance.

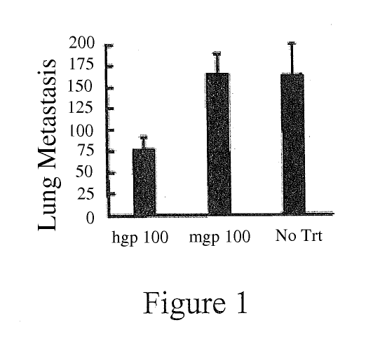

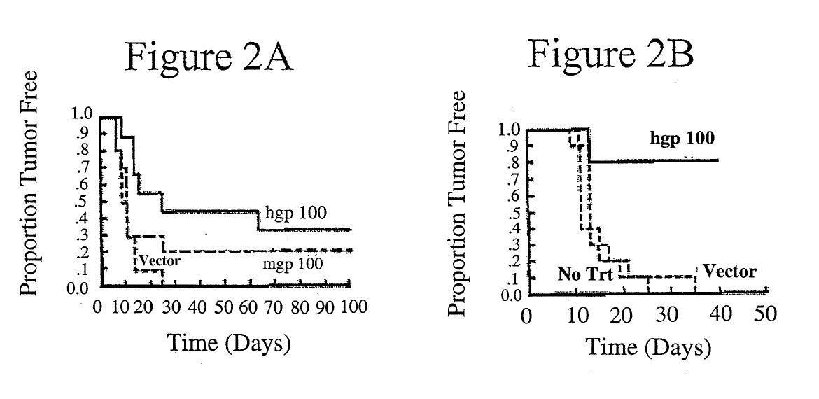

DNA immunization with xenogeneic hgp100 decreased lung metastasis by ˜50% as shown in FIG. 1. This immunization also increased tumor-free survival, as shown in FIGS. 2A-B (FIG. 2B shows a repeat of the experiment not including mgp100). Immunization with hgp100 3 times increased survival compared to mice immunized once and those with no treatment, as shown in FIG. 2C. Addition of mouse granulocyte-macrophage colony stimulating factor (GM-CSF) did not improve survival, as shown in FIG. 2D.

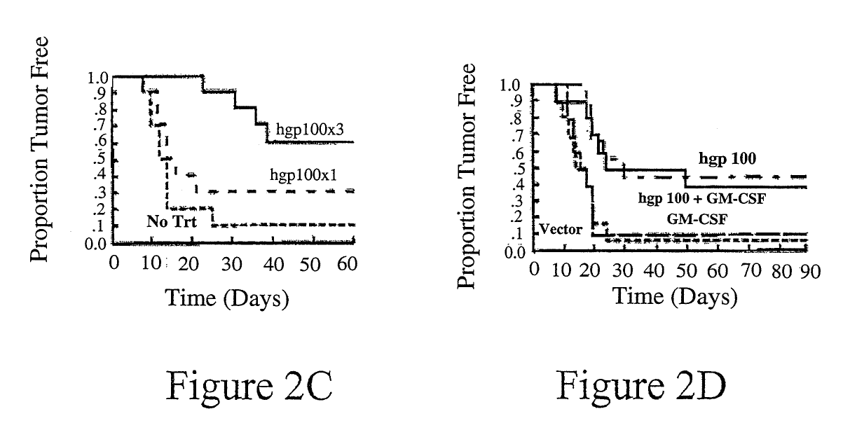

Depigmentation was measured by dividing the abdominal region into quadrants, and scoring a quadrant as depigmented when greater than 50% of hairs were depigmented. Depigmentation indicates the induction of autoimmunity against melanocytes. Depigmentation was observed in 7 of 10 mice that were immunized with hgp100, but not in any immunized with mgp100, empty vector, or untreated, as seen in FIG. 3.

### Example 2

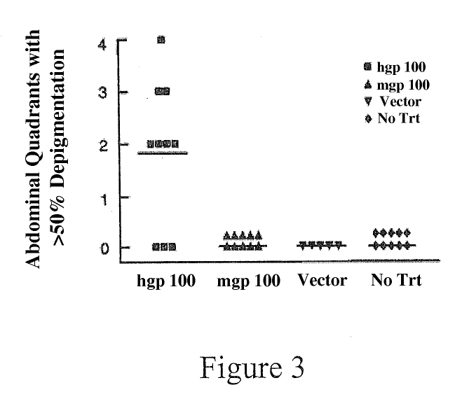

Using the same conditions described above, T-cells from immunized mice were obtained and tested for the presence of mouse peptide-specific cytotoxic T-lymphocyte (CTL) response. CTL response was detected after immunization only in hgp100 immunized mice. In addition, mice immunized with hgp100 showed greater numbers of CD8+ T-cells responding to mgp100 fragments shown by Elispot assay, as shown in FIG. 4A-B.

### Example 3

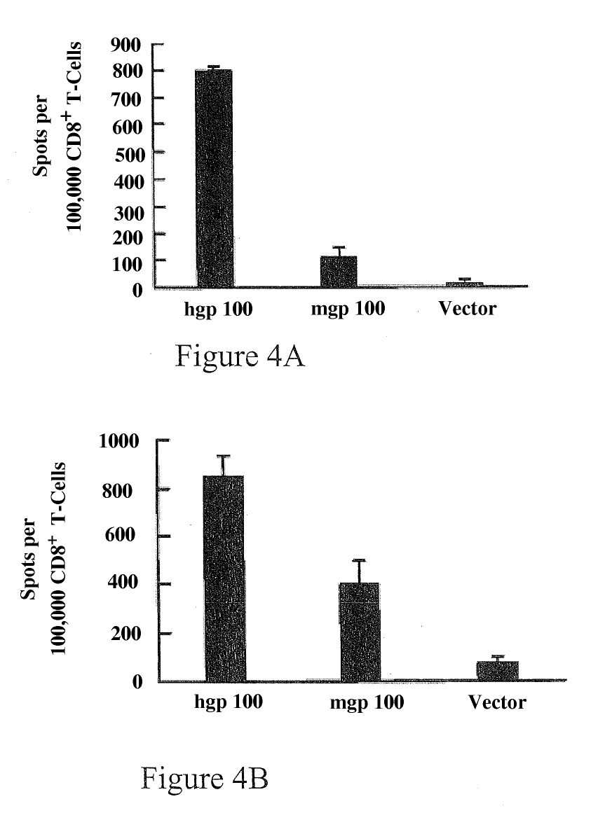

Mouse and human cDNA were introduced into a vector and a group of 19 human melanoma patients were injected with either xenogeneic mouse gp100 or human gp100 at three dosages (100, 500, or 1500 μg) every three weeks for three doses. After the first three doses, patients were immunized with gp100 from the other species. Five patients developed CD8+ cells binding gp100 fragments. This was determined by multi-parametric flow cytometry at Baseline, Cross-over, and Post-Vaccine. Representative examples are shown in FIG. 5. Fluorochromes used were a HLA-A*201-PE-labeled tetramer loaded with gp100 fragments and APC-AF750-CD8.

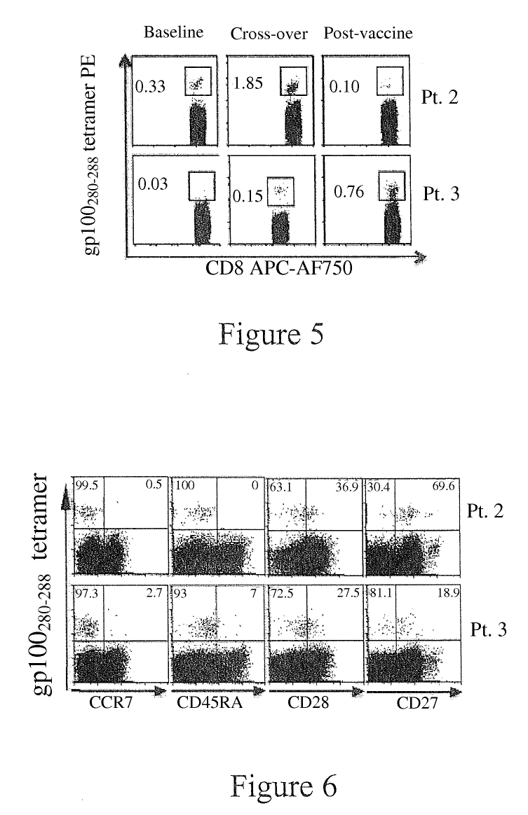

The CD8+ T-cells from these five patients were also examined for chemokine receptor 7. All five patients were CCR7^{lo} and CD45RA^{lo}. Of these, two were CD27^{hi} CD28^{lo} and the others were CD27^{hi} CD28^{int}. Representative dot plots are shown in FIG. 6.

Intracellular cytokine staining was performed on CD8+ cells. One patient was found to have an increase in CD8+IFN-γ+ cells after immunization. This is reflected in FIG. 6.

There was no statistically significant difference observed in the order or dosage level of the immunization vaccine given.

### SEQUENCES

The sequence of Human gp100 is known from Adema G et al., Molecular Characterization of the Melanocyte Lineage-specific Antigen gp100, 269 J. BIOL. CHEM. 20126 (1994).

| Seq. ID No. 1 - human gp100 | |
|---|---|
| 1 | agtgcctttg gttgctggag ggaagaacac aatggatctg gtgctaaaaa gatgccttct |
| 61 | tcatttggct gtgataggtg ctttgctggc tgtgggggct acaaaagtac ccagaaacca |
| 121 | ggactggctt ggtgtctcaa ggcaactcag aaccaaagcc tggaacaggc agctgtatcc |
| 181 | agagtggaca gaagcccaga gacttgactg ctggagaggt ggtcaagtgt ccctcaaggt |
| 241 | cagtaatgat gggcctacac tgattggtgc aaatgcctcc ttctctattg ccttgaactt |
| 301 | ccctggaagc caaaaggtat tgccagatgg gcaggttatc tgggtcaaca ataccatcat |
| 361 | caatgggagc caggtgtggg gaggacagcc agtgtatccc caggaaactg acgatgcctg |
| 421 | catcttccct gatggtggac cttgcccatc tggctcttgg tctcagaaga gaagctttgt |
| 481 | ttatgtctgg aagacctggg gccaatactg gcaagttcta gggggcccag tgtctgggct |
| 541 | gagcattggg acaggcaggg caatgctggg cacacacacc atggaagtga ctgtctacca |
| 601 | tcgccgggga tcccggagct atgtgcctct tgctcattcc agctcagcct tcaccattac |
| 661 | tgaccaggtg cctttctccg tgagcgtgtc ccagttgcgg gccttggatg gagggaacaa |
| 721 | gcacttcctg agaaatcagc ctctgacctt tgccctccag ctccatgacc ccagtggcta |
| 781 | tctggctgaa gctgacctct cctacacctg ggactttgga gacagtagtg gaaccctgat |
| 841 | ctctcgggca cttgtggtca ctcatactta cctggagcct ggcccagtca ctgcccaggt |
| 901 | ggtcctgcag gctgccattc ctctcacctc ctgtggctcc tccccagttc caggcaccac |
| 961 | agatgggcac aggccaactg cagaggcccc taacaccaca gctggccaag tgcctactac |
| 1021 | agaagttgtg ggtactacac ctggtcaggc gccaactgca gagccctctg gaaccacatc |
| 1081 | tgtgcaggtg ccaaccactg aagtcataag cactgcacct gtgcagatgc caactgcaga |
| 1141 | gagcacaggt atgacacctg agaaggtgcc agtttcagag gtcatgggta ccacactggc |
| 1201 | agagatgtca actccagagg ctacaggtat gacacctgca gaggtatcaa ttgtggtgct |
| 1261 | ttctggaacc acagctgcac aggtaacaac tacagagtgg gtggagacca cagctagaga |
| 1321 | gctacctatc cctgagcctg aaggtccaga tgccagctca atcatgtcta cggaaagtat |
| 1381 | tacaggttcc ctgggccccc tgctggatgg tacagccacc ttaaggctgg tgaagagaca |
| 1441 | agtccccctg gattgtgttc tgtatcgata tggttccttt tccgtcaccc tggacattgt |
| 1501 | ccagggtatt gaaagtgccg agatcctgca ggctgtgccg tccggtgagg gggatgcatt |
| 1561 | tgagctgact gtgtcctgcc aaggcgggct gcccaaggaa gcctgcatgg agatctcatc |
| 1621 | gccagggtgc cagccccctg cccagcggct gtgccagcct gtgctaccca gcccagcctg |
| 1681 | ccagctggtt ctgcaccaga tactgaaggg tggctcgggg acatactgcc tcaatgtgtc |
| 1741 | tctggctgat accaacagcc tggcagtggt cagcacccag cttatcatgc ctggtcaaga |
| 1801 | agcaggcctt gggcaggttc cgctgatcgt gggcatcttg ctggtgttga tggctgtggt |
| 1861 | ccttgcatct ctgatatata ggcgcagact tatgaagcaa gacttctccg taccccagtt |
| 1921 | gccacatagc agcagtcact ggctgcgtct accccgcatc ttctgctctt gtcccattgg |
| 1981 | tgagaatagc cccctcctca gtgggcagca ggtctgagta ctctcatatg atgctgtgat |
| 2041 | tttcctggag ttgacagaaa cacctatatt tcccccagtc ttccctggga gactactatt |
| 2101 | aactgaaata aatactcaga gcctgaaaaa aaaaaaaaaa aaa |

The cDNA for mouse gp100 was isolated and cloned into a vector in Adema G, et al, Melanocyte Lineage-Specific Antigens Recognized by Monoclonal Antibodies NKI-beteb, HMB-50, and HMB-45 are Encoded by a Single cDNA, 143 Amer. J. of Pathology 1579 (1993).

| Seq. ID No. 2 - mouse gp100 | |
|---|---|
| 1 | atgggtgtcc agagaaggag cttccttccc gtgcttgtgc tgagtgctct gctggctgtg |
| 61 | ggggccctag aaggatccag gaatcaggac tggcttggtg tcccaagaca acttgtaact |
| 121 | aaaacctgga acaggcagct gtaccccgag tggacagagg tgcaggggtc taactgctgg |
| 181 | agaggtggcc aggtatctct gagggtcatt aatgatgggc ctacactggt tggtgcaaat |
| 241 | gcctcctttt ccattgccct gcacttccct ggaagtcaaa aggtactacc ggatggtcag |
| 301 | gttatctggg ccaacaacac catcatcaat gggagccagg tgtggggagg acagccagtg |
| 361 | tatccacagg agcctgatga tgcctgtgtc ttccctgacg gtggaccctg cccatctggt |
| 421 | cctaaacctc cgaagagaag ctttgtttat gtttggaaga cctggggaaa atactggcaa |
| 481 | gttctggggg gtccagtgtc caggctgagc attgctacgg gccacgcaaa gctgggcaca |
| 541 | cacacaatgg aagtgactgt ctaccaccga cggggttccc agagctacgt gccccttgct |
| 601 | cacgccagtt caaccttcac cattactgac caggtacctt tctccgtgag tgtgtcccag |
| 661 | ctacaggcct tggacggaga gaccaagcac ttcctgagaa atcatcctct catctttgcc |
| 721 | cttcagctcc acgaccccag tggttatttg gccgaggccg acctctccta cacatgggac |
| 781 | tttggagatg gtactgggac cctgatctct cgggcacttg atgtcactca cacttacctg |
| 841 | gagtcgggct cagtcactgc ccaggtggta ctgcaggctg ccattcctct tgtttcctgt |
| 901 | ggttcctccc cagtcccggg taccacagat ggctacatgc caactgcaga agcacctgga |
| 961 | accacatcta ggcaaggaac cactacaaaa gttgtgggta ctacacctgg ccagatgcca |
| 1021 | actacacagc cctctggaac cacagttgta caaatgccaa ccacagaggt cacagctact |
| 1081 | acatctgagc agatgctgac ctcagcggtc atagatacca cactggcaga ggtgtcaact |
| 1141 | acagagggta caggtaccac acccacaagg ccttctggaa ccaccgttgc acaagcaaca |
| 1201 | accacagagg gtccagatgc cagcccattg ctgcccacac aaagttctac agggtccatt |
| 1261 | agccctctac tggatgacac cgacaccata atgcttgtga agagacaagt tcccctggac |
| 1321 | tgtgttctat atcgatatgg ttctttctcc ctcgccctgg acattgtcca gggtattgaa |
| 1381 | agtgctgaga tcctgcaggc tgtgccattc agtgaagggg atgcatttga gctgactgtg |
| 1441 | tcctgccaag gcgggctacc caaggaagcc tgtatggaca tttcatcacc agggtgccag |
| 1501 | ccccctgccc agaggctgtg ccagtctgtt ccaccgagcc cagactgcca gctggttcta |
| 1561 | caccaagtgc tgaaaggtgg ctcagggaca tattgcctca atgtgtcttt ggctgacgcc |
| 1621 | aacagcctgg cagtggccag cacccaactt gttgttcctg gtcaagacgg tggccttggg |
| 1681 | caggctccct tgcttgtagg tatcttgctg gtgttggtgg ctgtggtcct tgcatctctg |
| 1741 | atacataggc atagacttaa gaagcagggc tcagtttccc aaatgccaca tggtagcact |
| 1801 | cactggctgc gcctgcctcc ggtcttccgc gcccgcggcc ttggagaaaa cagcccgctc |
| 1861 | ctcagtggac agcaggtctg a |

All references cited herein are incorporated by reference.

We claim:

1. A method for stimulating an immune response to a tissue expressing gp100 in a subject individual of a first species, comprising administering to the subject individual an immunologically-effective amount of xenogeneic or xenoexpressed gp100 antigen.

2. The method according to claim 1, wherein the subject individual of the first species is human.

3. The method of claim 1, wherein the gp100 antigen is a xenogeneic gp100 antigen derived from a source selected from the group consisting of rodents, dogs, cats, cows, and sheep gp100 antigen.

4. The method of claim 1, wherein the step of administering is achieved by immunization with DNA encoding a xenogeneic gp100 antigen.

5. The method of claim 4, wherein the DNA immunization is achieved by immunization with liposomes comprising DNA encoding the xenogeneic gp100 antigen.

6. The method of claim 4, wherein the DNA immunization is achieved by immunization with gold particles coated with DNA encoding the xenogeneic gp100 antigen.

7. The method of claim 4, wherein the DNA encoding the gp100 antigen is an expression vector encoding the gp100 antigen.

8. The method of claim 1 wherein the immune response is a cellular or humoral response.

9. The method of claim 8 wherein the amount of xenogeneic or xenoexpressed gp100 antigen is sufficient to provide immunoprotection against growth of tumors expressing gp100.

10. The method of claim 1 wherein the amount of xenogeneic or xenoexpressed gp100 antigen is sufficient to provide immunoprotection against growth of tumors expressing gp100.

11. The method of claim 1, wherein the step of administering is achieved by immunization with a construct comprising DNA encoding a xenogeneic gp100 antigen, said construct resulting in expression of the xenogeneic gp100 antigen in the subject individual.

12. The method of claim 1, wherein the subject individual has melanoma.

13. The method of claim 12, wherein the step of administering is achieved by immunization with a construct comprising DNA encoding a xenogeneic gp100 antigen, said construct resulting in expression of the xenogeneic gp100 antigen in the subject individual.
